(19) United States
(12) Patent Application Publication
(10) Pub. No.: US 2007/0275456 A1
(43) Pub. Date: Nov. 29, 2007

(54) Diagnostic Testing Apparatus

(76) Inventors: David Ian Marlborough, Balgowlah (AU); Matthew Charles Archer Durack, Glebe (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/594,658

(22) PCT Filed: Apr. 5, 2005
(86) PCT No.: PCT/AU05/00490

§ 371 Date: Jul. 25, 2007

(30) Foreign Application Priority Data
Apr. 5, 2004 (AU) 2004901816

(51) Int. Cl. C12M 1/34

(52) U.S. Cl. 435/287.2

(57) ABSTRACT

An apparatus for use in an assay process includes a pre-incubation chamber (10) defining a plurality of wells (14) having a porous base (18). The pre-incubation chamber (10) is attachable to a housing (12) carrying a porous membrane (32) disposed above and touching a body of absorbent material (36). A pattern of capture analyte is carried on the porous membrane (32). Pins depending from the underside (20) of the pre-incubation chamber locate in holes defined in corners on the top of the housing to locate the chamber housing in a set position on top of the housing such that capture analyte is disposed under the bases of the wells of the pre-incubation chamber.

### FIELD OF THE INVENTION

This invention relates to improved diagnostic testing apparatus.

### BACKGROUND OF THE INVENTION

Enzyme-linked immunosorbent assay (ELISA) is a well known assay method in which antigens or antibodies are detected by means of an enzyme chemically coupled either to an antibody specific for the antigen or to anti-Ig which in turn will bind to the specific antibody. Either the antigen, or the antibody to be detected, is attached to the surface of a small container or to plastic beads and the specific antibody is allowed to bind in turn. The amount bound is subsequently measured by addition of a substrate for an enzyme which develops a colour when hydrolysed.

The steps involved in a typical ELISA procedure are, assuming that a coated plate with a target antigen is available, as follows.

1) The sample and a reference standard are diluted and the diluted samples are aliquotted into a microwell array plate.

2) The microwell array plate is incubated for between 10 minutes to 2 hours at ambient temperature or at temperatures of up to 37° C.

3) The plate is rigorously washed to remove the samples to ensure complete removal of unbound analyte and/or any interfering agent.

4) Next, diluted primary antibody is added, typically unconjugated, but in some cases carrying a marker probe directly.

5) Step 2, the incubation of the plate, is then repeated.

6) Step 3, the washing step, is then repeated.

7) Where step 4 is the addition of an unconjugated primary antibody, a secondary antibody is added conjugated to a marker probe.

8) The incubation step is repeated where secondary antibody has been added.

9) The washing step is repeated where secondary antibody has been added.

10) A substrate for the marker probe is added, if the marker is an enzyme.

11) A stopping reagent is added at a specified time interval: the time can vary depending upon the nature of the secondary antibody binding.

12) The results are measured in a dedicated plate-reader.

In an ideal ELISA analysis where incubation steps can be limited to 10 minutes and the primary antibody is conjugated to a probe, there are a minimum of seven steps and a time span of at least 30 minutes to conduct the procedure. More typically, the procedure requires eleven steps and takes two and a half to three hours to complete.

The present invention seeks to provide a more rapid diagnostic test apparatus but which still provides a degree of quantitative precision.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

In a first aspect of the present invention there is provided an apparatus for use in an assay process comprising:

a pre-incubation chamber defining a plurality of wells, the wells having a porous base; and

a housing carrying a porous membrane disposed above and touching a body of absorbent material, and

wherein a pattern of capture analyte is carried on the porous membrane further including means for locating the pre-incubation chamber on top of the housing such that capture analyte is disposed under the base of the wells of the pre-incubation chamber.

The housing may be separate from the pre-incubation chamber or may be attached to it in an arrangement in which the porous bases of the wells may be moved towards and away from the membrane.

Typically the separate housing defines a rectangular frame in which the membrane and body of absorbent material locate. The membrane is typically also rectangular and is substantially the same size and shape as the frame so that the when inserted in the frame, the location of the frame defines the location of the membrane.

The capture analyte may be deposited in strips, in lines or an array of dots, on the porous membrane by printing. Each strip may comprise several closely spaced lines of different capture analyte so that, in use, each well can be used to test for several reagents simultaneously.

The porous base may comprise a frit or porous plug. The plug may depend below the underside of the pre-incubation chamber.

The means for locating the pre-incubation chamber on top of the housing may comprise pins depending from the underside of corners of the pre-incubation chamber, and corresponding holes defined corners of the housing.

Terminology used in the art to describe diagnostic apparatus and methods can be confusing and in some cases similar terms can be used to describe different components of the test. For the avoidance of doubt, in the foregoing description, the following terms used in the description are defined as follows. The term “reagent” is used to refer to the compound protein or other reagent which is to be detected by the assay. The term “capture analyte” is used to refer to a compound which is bound to a membrane and to which the reagent will bind. The term “detection analyte” is used to refer to a compound which will also bind to the reagent and which carries a tracer or some other element whose presence may be detected, typically visually detected, whether under visible light or fluorescent light.

### BRIEF DESCRIPTION OF THE DRAWINGS

A specific embodiment of the present invention will now be described, by way of example only, and with reference to the accompanying drawings in which:

FIG. 1 illustrates a pre-incubation chamber and a flow-through cassette of a diagnostic test apparatus;

FIG. 2 illustrates an absorbent pad and membrane which is carried by the flow-through cassette;

FIG. 3 shows a section through a pre-incubation chamber along a vertical plane;

FIG. 3a in an enlarged view of part of the section shown in FIG. 3; and

FIG. 4 illustrates the fitting of the pre-incubation chamber to the flow-through cassette.

### DETAILED DESCRIPTION OF A PREFERRED EMBODIMENT

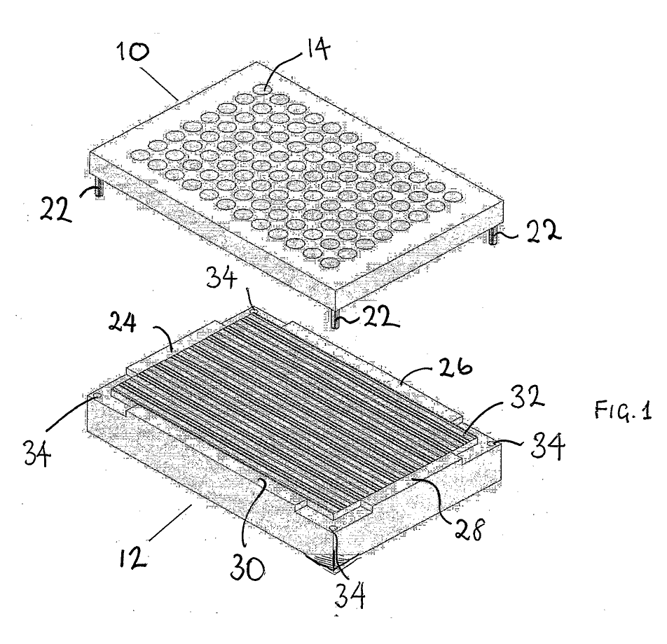

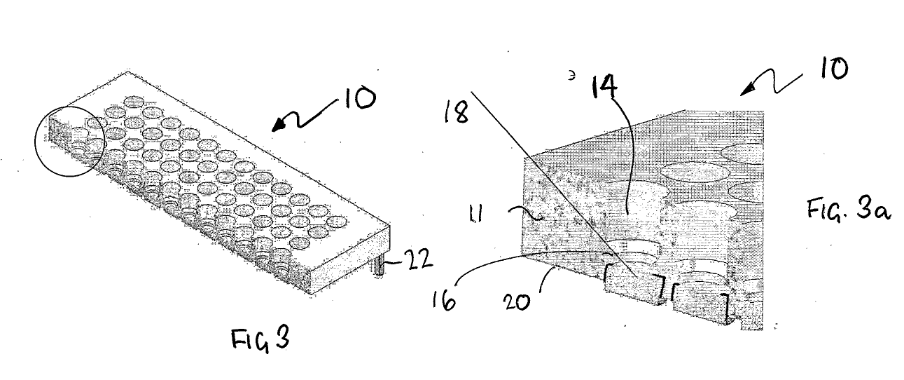

Referring to the drawings, FIG. 1 illustrates two components of a diagnostic apparatus being a pre-incubation chamber 10 and a housing in the form of a flow-through cassette 12. The pre-incubation chamber is in the form of a moulded rectangular block 11 made from a plastics material which defines ninety-six wells 14 arranged in twelve rows of eight wells. With reference to FIGS. 3 and 3a, each well is generally cylindrical having a generally constant circular horizontal cross section for the majority of its depth but defining a reduced diameter portion 16 where the well constricts, below which a polyethylene frit 18 in the form of a porous plug is press fitted. The lowermost part of the plug 18 depends below the underside 20 of the block 11. As is best seen in FIG. 1 a pin 22 depends from each corner of the pre-incubation chamber 10.

The flow-through cassette 12 comprises a moulded block made from a plastics material which includes four raised areas which define four sides 24, 26, 28, and 30 of a rectangular frame provided to receive and locate a rectangular membrane and pad assembly 32. A hole 34 is defined at each corner of the pre-incubation chamber. The holes are located and configured to receive the depending pins 22 of the pre-incubation chamber 10.

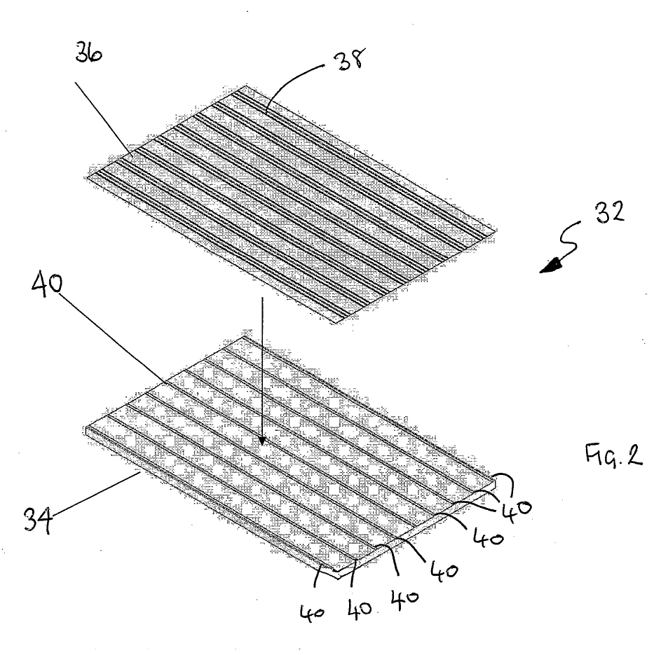

With reference to FIG. 2, the pad and membrane assembly 32 comprise an absorbent pad 34 which is typically made from absorbent paper, and a printed nitrocellulose membrane 36. Eight strips 38 of antibodies (capture analytes) are defined on the top of the membrane. These are printed on the membrane using syringe pumps. Each strip comprises three closely spaced lines of antibodies making twenty four rows of antibodies in total. Each strip of three antibodies includes three lines of different capture analytes and each strip includes the same three capture analytes. Each strip can detect a different reagent. More strips could be used to detect more reagents.

The nitrocellulose membrane is attached to the pad 34 by gluing. As shown in FIG. 2, lines of glue 40 are defined on the upper surface of the pad. The lines of glue are positioned so as to be located either side of the strips 38 of printed antibodies when the membrane is glued onto the pad, so as not to affect the results of the assay. The lines of glue may also be printed onto the pad 34 using syringe pumps.

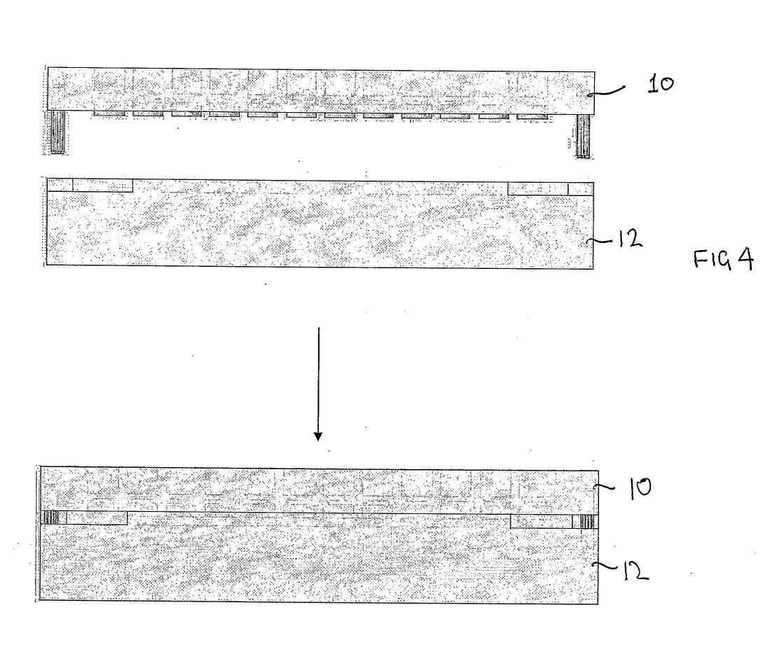

With reference to FIG. 4, in use, the sample which contains the reagent to be detected by the diagnostic test and the detection analyte—a colloidal immunogold conjugate, is loaded into the wells 14 of the pre-incubation chamber using an automated liquid handling means. A short pre-incubation step then takes place while the detection analyte binds to any reagent in the sample. This typically takes about two minutes.

As an alternative to adding the colloidal immunogold conjugate, the conjugate can be immobilised on the plug 18 or in a layer above the plug but in contact so that the sample can extract immunogold and then flow through into contact with the membrane 36.

Next, the pre-incubation chamber 10 is fitted onto the flow-through cassette 12 using the pins 22 and holes 34 to ensure that the chamber locates in the correct position, with the strips of antibodies located below the plugs 18 defining the bases of the wells. This is illustrated in FIG. 4. At this stage, the porous plugs 18 at the base of the wells of the pre-incubation chamber contact the nitrocellulose membrane 36 and the sample in the wells of the pre-incubation chamber is sucked through the absorbent pad 32. Any reagents which bind to the antibodies on the printed nitrocellulose membrane, will be captured by those antibodies as the sample is sucked through into the absorbent pad.

After filtration of the sample has taken place, carefully controlled quantities of washing buffer can be aliquotted into the wells of the pre-incubation chamber. The housing 10 is then removed and the flow-through cassette is placed in a dedicated reader to check the results of the assay.

In the above referenced process, there are only seven steps in total and the process takes about eight minutes to carry out. In the variant in which the immunogold conjugate is immobilised on the plug 18, the step of adding colloidal immunogold conjugate to the sample, is omitted and there is a small time saving of around 10-20 seconds.

The above compare with a typical ELISA test measurement which takes 1.5 to 2 hours, providing significant time savings.

A further advantage over the ELISA method is that more than one analyte can be detected in a well by striping more than one capture analyte line, for example, in the printed nitrocellulose membrane shown in FIG. 2, there are three analyte lines shown for each well.

In a variant, a cover, not shown, may be provided which can snap-fit over the pre-incubation chamber 10 during processing to reduce the chances of any sample in the wells of the pre-incubation chamber being spilt and for reducing the chances of splash-back.

In a further variant, it is envisaged that means to aspirate excess material from the underside of the pre-incubation chamber 10 will be provided. For example the bottom plate 12 may be designed with a space beneath the porous membrane that can be attached to a vacuum pump.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An apparatus for use in an assay process comprising: a pre-incubation chamber defining a plurality of wells, the wells having a porous base; and a housing carrying a porous membrane disposed above and touching a body of absorbent material, and wherein a pattern of capture analyte is carried on the porous membrane further including means for locating the pre-incubation chamber on top of the housing such that capture analyte is disposed under the base of the wells of the pre-incubation chamber.

2. An apparatus as claimed in claim 1 wherein the housing is separate from the pre-incubation chamber.

3. An apparatus as claimed in claim 1 wherein the housing is attached to the pre-incubation chamber in an arrangement in which the porous bases of the wells may be moved towards and away from the membrane.

4. An apparatus as claimed in claim 2 wherein the separate housing defines a rectangular frame in which the membrane and body of absorbent material locate and the membrane is also rectangular and is substantially the same size and shape as the frame so that the when inserted in the frame, the location of the frame defines the location of the membrane.

5. An apparatus as claimed in claim 1 wherein the capture analyte is deposited in strips, in lines or in an array of dots, on the porous membrane by printing.

6. An apparatus as claimed in claim 5 wherein each strip, comprises several closely spaced lines of different capture analyses so that, in use, each well can be used to test for several reagents simultaneously.

7. An apparatus as claimed in claim 1 wherein the porous base comprises a frit or porous plug. which depends below the underside of the pre-incubation chamber.

8. An apparatus as claimed in claim 2 wherein the means for locating the pre-incubation chamber on top of the housing comprise pins depending from the underside of corners of the pre-incubation chamber and holes defined in corners of the housing.
